(19) United States
(12) Patent Application Publication
(10) Pub. No.: US 2015/0206409 A1
(43) Pub. Date: Jul. 23, 2015

(54) SYSTEM, METHOD, SOFTWARE APPLICATION AND DATA SIGNAL FOR DETERMINING MOVEMENT

(71) Applicant: Adelaide Research & Innovation Pty Ltd, Adelaide (AU)

(72) Inventors: Renuka Visvanathan, Linden Park (AU); Rankothge Damith Chinthana Ranasinghe, Melrose Park (AU)

(21) Appl. No.: 14/417,583

(22) PCT Filed: Jul. 29, 2013
(86) PCT No.: PCT/AU2013/000838

§ 371 Date: Jan. 27, 2015

(30) Foreign Application Priority Data
Jul. 27, 2012 (AU) 2012903239

(51) Int. Cl. G08B 21/04; A61B 5/11

(52) U.S. Cl. CPC G08B 21/0423; A61B 5/11; G08B 21/043

(57) ABSTRACT

A method, system, application and data signal for determining movement is described herein. In an example, the system may include a processing hardware set and computer readable storage device medium, the processing hardware set adapted to be structured, connected and/or programmed to run program instructions stored on the computer readable storage medium. The program instructions may include at least one receiving module adapted to receive movement data indicative of movement from a remote device, at least one processing module adapted to process the moving data to determine a type of movement, and at least one alerting module adapted to provide an alert in the event that the type of movement falls within at least one predetermined type.

### BACKGROUND

1. Field

The example embodiments in general are directed to a system, method, software application and data signal for determining movement. The device, system, method, software application and data signal finds particular, but not exclusive, use in the monitoring of patients and objects in a hospital, aged care or other supervised environment, where it is important to track not only the location, but also the movement and change in position of a patient or object.

2. Related Art

Persons who are injured, weakened (due to disease, such as a stroke, for example) or aged are more prone to accidents or falls. Such accidents or falls can cause injury, which can result in hospital stays or the need for ongoing monitoring and treatment. More importantly, accidents or falls can lead to a loss of independence, a decline in health status and the development of psychological consequences such as anxiety, depression and loss of confidence.

Moreover, it is not only the person who falls who is affected by the fall. Where a patient in a hospital or a person in an aged care facility is injured, staff and family can feel fear, guilt and anxiety. These feelings can result in defensive actions being taken by the staff or family members, which may contribute to poorer care, more conflict and a rise in complaints, coroner's inquests and litigation.

Falls in Australian hospitals alone are estimated to increase the total number of hospital bed days by 886,000 per year. Such alarming figures will only increase over time due to an aging population in many countries.

### SUMMARY

An example embodiment of the present invention is directed to a computer system for determining movement. The system may include a processing hardware set and a computer readable storage device medium, the processing hardware set adapted to be structured, connected and/or programmed to run program instructions stored on the computer readable storage medium. The program instructions may include at least one receiving module adapted to receive movement data indicative of movement from a remote device, at least one processing module adapted to process the moving data to determine a type of movement, and at least one alerting module adapted to provide an alert in the event that the type of movement falls within at least one predetermined type.

Another example embodiment is directed to a method for determining movement. The method may include receiving movement data indicative of movement from at least one remote device, and processing the movement data to determine the type of movement. In the event that the type of movement falls within at least one predetermined type, an alert is provided. The movement data includes acceleration data and is useable to calculate a motion vector indicative of the movement of the remote device. Each of the receiving, processing, and providing steps is performed by computer software adapted to run on computer hardware.

Another example embodiment is directed to a set of machine readable instructions and associated data, stored on a storage device in a manner more persistent than a signal in transit. The set may include at least one receiving module programmed to receive movement data indicative of movement from a remote device, at least one processing module programmed to process the moving data to determine a type of movement, and at least one alerting module programmed to provide an alert in the event that the type of movement falls within at least one predetermined type.

### BRIEF DESCRIPTION OF THE DRAWINGS

Example embodiments will become more fully understood from the detailed description given herein below and the accompanying drawings, wherein like elements are represented by like reference numerals, which are given by way of illustration only and thus are not limitative of the example embodiments herein.

FIG. 1 is a diagram illustrating a system in accordance with an embodiment of the present invention.

FIG. 2 is a diagram illustrating the calculation of a velocity vector in accordance with an embodiment of the present invention.

FIG. 3a is a diagram illustrating the relative positioning of RFID readers utilised to calculate a transition (walking through a door) in accordance with an embodiment of the invention.

FIG. 3b is a graph of collected and processed data illustrating a pattern indicative of a person walking through a door, in accordance with an embodiment of the invention.

FIG. 4 is a graph of collected and processed data illustrating a pattern indicative of a person transitioning through a sit/stand movement, in accordance with an embodiment of the invention.

FIG. 5 is a graph of collected and processed data illustrating a pattern indicative of a person walking without a walking aid, in accordance with an embodiment of the invention.

FIG. 6 is a schematic of a computing system in accordance with an embodiment of the invention.

FIG. 7 is a diagram illustrating a system in accordance with an embodiment of the invention.

FIG. 8 is a diagram illustrating an algorithm developed in accordance with an embodiment of the invention.

FIGS. 9 and 10 are diagrams illustrating survey results collected to demonstrate the effectiveness of an embodiment of the invention.

FIG. 11 is a diagram illustrating a test layout of antennas in a room in accordance with an embodiment of the invention.

FIG. 12 is an automatically generated ‘visual cue’ (bed side poster) using the HIT tool in accordance with an embodiment of the invention.

FIG. 13 is a diagram illustrating a system in accordance with an embodiment of the invention.

FIG. 14 is a flowchart illustrating a process flow in accordance with an embodiment of the invention.

FIGS. 15a and 15b are screenshots illustrating a user interface in accordance with an embodiment of the present invention.

### DETAILED DESCRIPTION

As will be appreciated by one skilled in the art, the example embodiments of the present invention may be embodied as a system, method, set of machine readable instructions and associate data in a manner more persistent than a signal in transit, or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer-readable medium(s) having computer readable program code/instructions embodied thereon.

In yet another embodiment, the computing system(s), method(s) and computer program product(s) as described in the example embodiments can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of the example embodiments.

Exemplary hardware that can be used for the example embodiments includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this invention is dependent on the speed and/or efficiency requirements of the system, the particular function, and the particular software or hardware systems or microprocessor or microcomputer systems being utilized.

Any combination of computer-readable media may be utilized. Computer-readable media may be a computer-readable signal medium or a computer-readable storage medium. A computer-readable storage medium may be, for example, but not limited to, an electronic, magnetic, optical, electromagnetic, infrared, or semiconductor system, apparatus or device, or any suitable combination of the foregoing. A non-exhaustive list of specific examples for a computer-readable storage medium would include at least the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing.

In the context of this document, a computer-readable storage medium may be any tangible medium that can contain or store a program for use by or in connection with an instruction execution system, apparatus or device. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Accordingly, the present invention foresees that a non-transitory computer readable information storage media having stored thereon information, that, when executed by a processor, causes the steps described in more detail hereafter in the example method(s) to be performed.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including but not limited to wireless, wireline, optical fiber cable, RF, etc., or any suitable combination of the foregoing.

The techniques described herein can be implemented in a distributed computing system that includes a back-end component, e.g., as a data server, and/or a middleware component, e.g., an application server, and/or a front-end component, e.g., a client computer having a graphical user interface and/or a Web browser through which a user can interact with an implementation of the invention, or any combination of such back-end, middleware, or front-end components. The components of the system can be interconnected by any form or medium of digital data communication, e.g., a communication network. Examples of communication networks include a local area network (“LAN”) and a wide area network (“WAN”), e.g., the Internet, and include both wired and wireless networks.

Computer program code for carrying out operations for aspects or embodiments of the present invention may be written in any combination of one or more programming languages, including an object oriented programming language such as JAVA®, SQL™, PHP™, RUBY™, PYTHON®, JSON, HTML5™, OBJECTIVE-C®, SWIFT™, XCODE®, SMALLTALK™, C++ or the like, conventional procedural programming languages, such as the “C” programming language or similar programming languages, any other markup language, any other scripting language, such as VBScript, and many other programming languages as are well known may be used.

The program code may execute entirely on a user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet using an Internet Service Provider).

Embodiments and aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer-implemented process such that the instructions which execute on the computer or other programmable apparatus provide processes for implementing the functions/acts specified in the flowchart and/or block diagram block or blocks.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

As used herein, the terms “program” or “software” are employed in a generic sense to refer to any type of computer code or set of computer-executable instructions that can be employed to program a computer or other processor to implement various aspects of the present invention as discussed above. Additionally, it should be appreciated that one or more computer programs that when executed perform methods of the example embodiments need not reside on a single computer or processor, but may be distributed in a modular fashion amongst a number of different computers or processors to implement various aspects of the example embodiments.

Data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

As used herein, the phrase “present invention” should not be taken as an absolute indication that the subject matter described by the term “is covered by either the claims as they are filed, or by the claims that may eventually issue after patent prosecution; while the term “present invention” is used to help the reader to get a general feel for which disclosures herein are believed as maybe being new, this understanding, as indicated by use of the term “present invention,” is tentative and provisional and subject to change over the course of patent prosecution as relevant information is developed and as the claims are potentially amended.

Unless the context requires otherwise, throughout the specification and claims that follow, the word “comprise” and variations thereof, such as “comprises” and “comprising,” are to be construed in an open, inclusive sense, that is, as “including, but not limited to.”

Reference throughout this specification to “one example embodiment” or “an embodiment” means that a particular feature, structure or characteristic described in connection with the embodiment is included in at least one embodiment. Thus, the appearances of the phrases “in one example embodiment” or “in an embodiment” in various places throughout this specification are not necessarily all referring to the same embodiment. Further, the particular features, structures or characteristics may be combined in any suitable manner in one or more example embodiments.

As used in this specification and the appended claims, the singular forms “a,” “an,” and “the” include plural referents unless the content clearly dictates otherwise. The term “or” is generally employed in its sense including “and/or” unless the content clearly dictates otherwise.

As used in the specification and appended claims, the terms “correspond,” “corresponds,” and “corresponding” are intended to describe a ratio of or a similarity between referenced objects. The use of “correspond” or one of its forms should not be construed to mean the exact shape or size.

In the drawings, identical reference numbers identify similar elements or acts. The size and relative positions of elements in the drawings are not necessarily drawn to scale.

### Overview of an Embodiment

As will be described in more detail hereafter, the example embodiments are directed to a method, computer system, software application, non-transitory computer readable information storage media, and to a set of machine readable instructions and associated data stored in a storage device, each adapted for determining movement. In an example, the movement data may include acceleration data, which may be utilised to calculate a motion vector indicative of the movement of the remote device. In an example, the type of movement is determined, in part, by analysing variations in the motion vector over a period of time.

In the example system, a receiving module is arranged to receive movement data from a plurality of remote devices and may receive the movement data as a radiofrequency signal. In an example, the receiving module incorporates a radiofrequency signal emitter arranged to send an activation signal to the remote device, such that where the remote device is a passive radiofrequency device, the remote device emits a radiofrequency signal encoding the movement data upon exposure to the activation signal.

In an example, the types of movements may include movements by a person or object. Where the predetermined types of movements are movements by a person, they may include movements likely to cause injury and movements likely to carry a high risk of injury when performed.

In an example, the receiving module receives the movement data indicative of movement from a remote device adapted so as to be wearable by a person. The remote device may be adhered, attached, or integrated into a wearable item, such as an item of clothing.

The example system may include a processing module selects a movement type from a group including walking through a doorway, sitting, standing, lying, getting up from a lying down position, and walking without a walking aid. However, it will be understood that the processing module may also be programmed to identify other movement types. In an example, the processing module may be arranged to calculate the radial velocity of the at least one remote device over a defined period of time and may be further configured to determine whether the direction of the radial velocity has changed over the defined period of time. Additionally, the processing module may be configured so as to classify the type of movement as walking through a doorway when the direction of the radial velocity has changed over the defined period of time, and to calculate an acceleration component in one predefined axis over a defined period of time and the predefined axis may be an axis substantially vertical relative to a ground surface. Further, the processing module may be adapted to analyze the acceleration component over a period of time to determine whether a pattern exists.

Moreover, and in an example, the processing module may be further configured to classify the type of movement as walking without a walking aid when the pattern of a first device does not correspond with the pattern of a second device, and to classify the type of movement as lying when the acceleration component is approximately zero and may additionally be arranged to calculate the angular displacement of the at least one remote device relative to a predefined axis over a defined period of time.

The processing module may also be configured to determine whether the angular displacement of the at least one remote device increases to a maximum value from a base level and subsequently returns to the base level, and to classify the type of movement as sitting when the angular displacement has changed over the defined period of time to the maximum state and subsequently returns to a base level.

The example system may include an alert module configured to send an alert to a person, wherein the alert is sent at defined intervals to the person until such time as the person satisfies a criterion. The system keeps a record of the amount of time elapsed between the sending of the alert to the person and the time at which the person satisfies the criterion. In one example, the receiving module receives identification data from the remote device and optionally, the alert module utilizes the identification data to, in part, determine the alert condition.

Accordingly, as to be further described in detail hereafter, the system comprises at least one receiving module arranged to receive movement data indicative of movement from a remote device, a processing module arranged to process the moving data to determine the type of movement, and an alert module arranged to provide an alert in the event that the type of movement falls within at least one predetermined type.

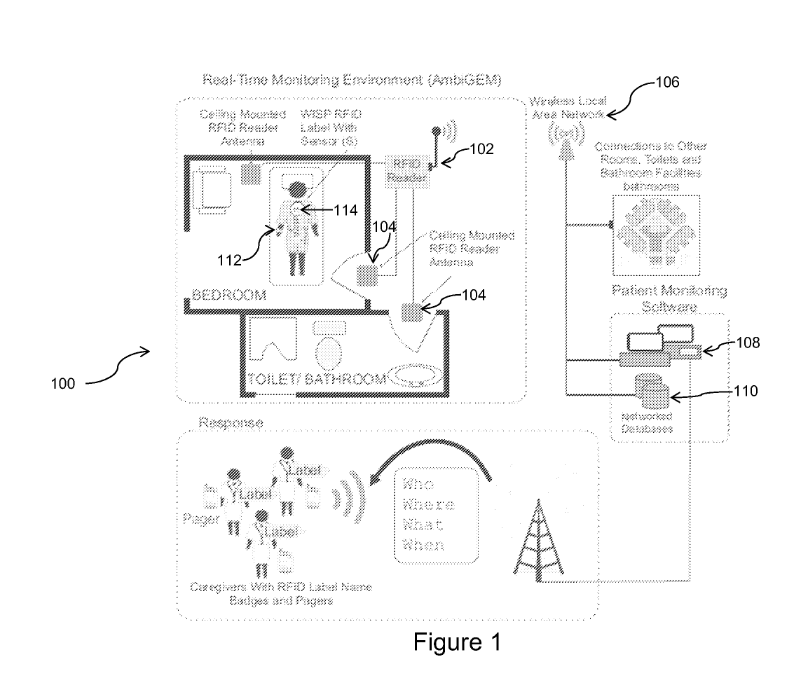

In more detail, with reference to FIG. 1, there is shown a schematic diagram which provides an overview of the components that make up a system 100 for determining movement (i.e. the real time monitoring of patient movement) in accordance with an embodiment of the invention. The system is described herein by way of example with reference to a hospital or aged care facility and is referred to as the AmbiGEM™ system.

It will be understood that the system may find use in any suitable environment where it is desirable to monitor high risk movement activities. While the example refers to a hospital or aged care facility, the system may also be used in individual homes to allow for people with dementia to live independently.

Moreover, the system may find use in industrial or commercial applications, to monitor workers who engage in high-risk activities that require compliance with occupational health and safety laws and regulations. In yet another example, the system may find use in high risk recreational activities, where participants in sporting activities are engaged in movement or actions that may pose a risk of injury.

The system includes a receiving module, which in the embodiment described herein, includes plurality of radiofrequency identification (RFID) readers 102 and associated antennas 104 that are connected via a wireless local area network (WLAN) 106. The RFID readers 102 communicate with a computing system 108 which in turn includes (or is connected to) a database 110. The computing system 108 includes a processing module which is arranged to execute monitoring software which receives data (information) via the wireless local area (WLAN) infrastructure 106. The computing system includes an appropriate interface (not shown) to allow direct interaction with the monitoring software. The monitoring software will be described in more detail below.

Patients (i.e. persons) 112 who are under the care of the facility (e.g. the aged care facility or hospital) and are physically located within the physical grounds of the facility (i.e. they are within the “environment” of the facility) are equipped with remote devices in the form of wearable Wireless Sensing and Identification (WISP) devices 114.

In turn, caregivers 116 carry pagers 118 or other mobile devices (such as mobile (cell) phones which are arranged to receive alerts generated by an alert module which is associated with or incorporated into the computing system.

Alerts are generated when the processing module executes the monitoring software (which includes an inference engine) to detect the occurrence of predetermined movement types, such as a high-risk action or a “fall”. That is, the monitoring software utilises the inference engine, which includes a series of algorithms to classify movement into one of a number of “types”. If the type detected falls into one of a predetermined number of categories of high-risk actions (which are described in more detail below), then the alert module is arranged to provide an alert to one or more caregivers 116.

Moreover, in one embodiment, caregivers wear RFID name badges to facilitate the automatic identification and localization of caregivers to monitor that an intervention is being administered or prevent activation of alert as caregiver supervision was already in place.

### WISP Devices

In more detail, the remote (WISP) devices 114 are Wireless Identification and Sensing devices which include passive (i.e. battery-less) Radio Frequency Identification (RFID) technology and a motion sensor, such as a tri-axial accelerometer. WISP devices are sometimes colloquially referred to as “tags”.

Known WISP devices are approximately 20 mm×20 mm in size and approximately 2 mm thick and include an antenna for transmitting and receiving radio frequency signals. WISP devices weigh approximately 2 grams.

As a WISP device is light and small in size, it is easily and generally undetectably incorporated into a number of different items, such as badges, “stickers”, clothing, and/or shoes or belts. It can also be attached or incorporated into any type of object, including walking aids, wheelchairs, etc.

WISPs are powered by harvested energy from radio waves transmitted from RFID readers. The harvested energy operates a 16-bit microcontroller (MSP430F2132) and a tri-axial accelerometer (ADXL330). The microcontroller can perform a variety of computing tasks, such as collecting (sampling) sensor data and reporting the sensor data to a remotely located receiving module, such as a RFID reader.

For completeness, it is noted that there are two different methods for transferring power from a RFID reader to the WISP device—magnetic induction and coupling to the electromagnetic (EM) waves transmitted by an RFID reader. The powering method depends on the distance of the WISP device to the RFID reader antennas where a distinction is made between a near field (energy storage field) and a far field (where electromagnetic wave propagation is dominant). Through various modulation techniques, data is also encoded onto the same transmitted signal from the reader to the WISP device and the received signal from the WISP device to the RFID reader. The embodiment described herein and the broader inventive concepts are equally capable of operating with both methods.

### WISP Attached to a Patient's Sternum

In one embodiment of the invention, a WISP tag is located over the sternum of a patient (user) at a location on top of their attire. It will be understood that the WISP tag may be incorporated in clothing or otherwise securely attached to the user.

### Mattress Attached WISP Method

In another embodiment, a WISP is attached to the side of the bed opposite the side of the bed most frequently used by the patient to get in or out of bed. This is done to avoid damage to the device or occlusion from the subject's body. The signal of interest corresponds to the acceleration readings of the z axis (zp), perpendicular to both gravity and the side of the mattress, in percentage values (where 50% is equivalent to 0 g) and its derivative z′p. If a patient lies or sits on the mattress, the change of the alignment of the sensor as a result of the deformation of the mattress during the activity causes a change in zp. It is known that if a patient keeps a static posture in bed or if the mattress is empty, the derivative of zp (z′p) remains in a defined value range. An algorithm was developed considering the changes in z′p and zp to identify a significant movement in bed as a PT out of a static state or significant movement made by the subject while remaining in a static state.

### Receiving Modules (RFID Readers)

In more detail, the receiving modules (in the form of RFID readers) are located at fixed locations with their antennas strategically placed to detect objects that incorporate a WISP device. The receiving modules further incorporate a module arranged to emit an electromagnetic field, such that power is transferred to WISP devices within a particular area around the RFID reader. RFID readers can read multiple co-located WISP devices simultaneously (up to several hundred WISP devices per second can be read by many known RFID systems). The reading distance ranges from a few centimetres to more than 10 meters, depending on the type of WISP device, the transmitted power of the RFID reader, antenna gain and interference from other radio frequency radiation.

Ultra High Frequency (UHF) RFID readers operate between 920 MHz and 926 MHz in Australia. Based on currently available studies, there are no known adverse effects from RFID readers operating in the UHF region on pace makers or implantable cardioverter-defibrillators, physiological monitors (such as electrocardiogram monitors) and intravenous pumps, making such RFID readers suitable for use in aged care or hospital environments.

In the embodiment described herein, the reader antenna configuration used is capable of communicating with conventional RFID tags (up to 10 metres away) and WISP devices (up to 3 metres away). However, it will be understood that different configurations arranged to operate at larger distances are within the purview of a person skilled in the art, and the example given herein should not be considered to be limiting on the broader inventive concept described and defined herein.

### Analysis and Identification of High Risk Activities by the Processing Module

Falls commonly occur around patients' or residents' beds, bathrooms and/or toilets or bathrooms. Consequently, high risk activities within an environment such as an aged care facility or a hospital that are likely to lead to a fall include but are not limited to:
1. entering a room or bathroom or toilet through a doorway;
2. getting up from a chair or sitting down in a chair;
3. getting into our or out of a bed; and
4. walking without the required walking aid.

The acceleration data and/or the resultant velocity vector received by the computing system from the receiving module (which in turn has received the data from a WISP device) is used to determine the movement type performed by the patient.

In particular, movement data is collected over a defined period of time and analysed using a number of techniques and/or algorithms. Each movement type is detected by determining the presence (or absence) of certain patterns in the movement data over a defined period of time. In the embodiment described herein, the algorithms utilised to determine the four particular movement types listed above are described in detail. It will be understood, however, that other relevant movement types are detected/detectable by the computing system.

### Algorithms for Detecting Movement Types

### Entering a Room Through a Doorway

In order to detect movement from one area to another (i.e. through a doorway), it is necessary to extract the velocity vector from the acceleration data provided by the WISP, to then in turn estimate the projection of the projection of the WISP velocity vector on to the line of sight between the WISP and the reader

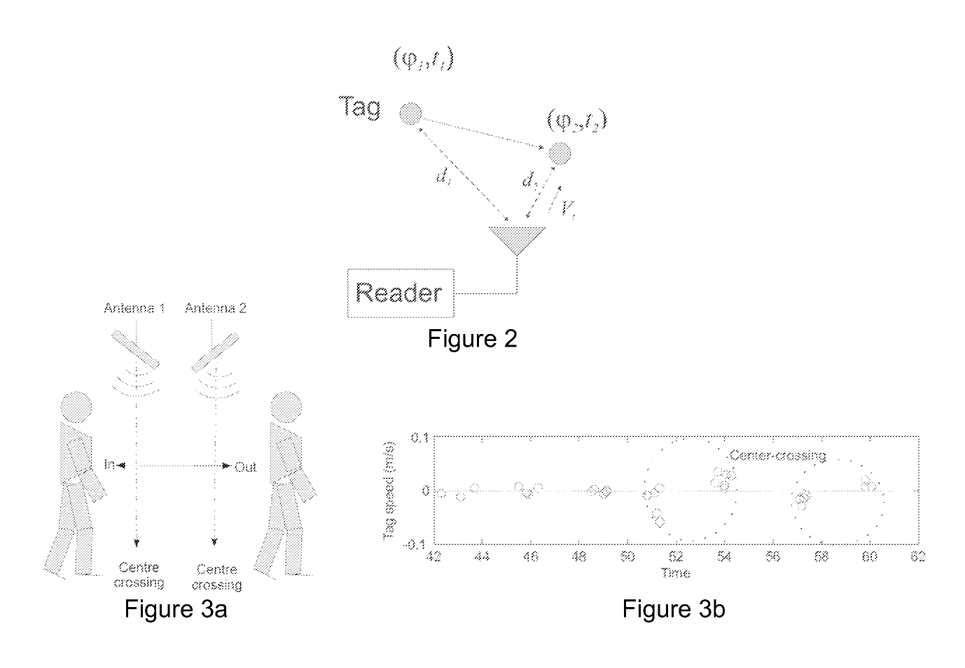

The projection of the WISP velocity vector on to the line of sight between the WISP and the RFID reader can be estimated by Time Domain Phase Difference of Arrival (TD-PDOA) measuring the phase of a tag at different time moments at the same frequency, as illustrated in FIG. 2. The difference of phase (φ₂−φ₁) at different times is measured and attributed to the path difference d₂−d₁. In turn, the radial velocity of the RFID tag is given by equation (1):

$${V}_{r} = \frac{\frac{1}{2} ( \frac{{ϕ}_{2} - {ϕ}_{1}}{x π} λ )}{{t}_{2} - {t}_{1}} = - \frac{c}{4 π f} \frac{Δϕ}{Δ t} ; \qquad ( 1 )$$

where λ=c/f (c is the speed of light and f is the frequency of the transmitted wave from the reader). The negative sign defines the direction of the radial velocity in the derivation as being opposite to the change in distance of the tag at times t₁ and t₂.

By analysing the radial velocity over time at two overhead antennae and determining the point at which the direction of the radial velocity changes from negative to positive, the time at which the person moves across a hypothetical “centre line” between the first and second antennas can be determined. This is shown at FIG. 3(a). Therefore by using two overhead antennae (as illustrated in FIG. 3(a), strategically placed at each side of a doorway entrance or other transition point, a person's traversing direction can be identified by analysing and comparing the centre-crossing evaluated by both antennae, as shown in FIG. 3(b).

In practice, to achieve an accurate and predictable result, the antennae of the two RFID readers are generally suspended from a ceiling (or a surface above “head height”) and the antenna of each RFID reader is leaned to an angle approximately 50 degrees from the vertical.

### Getting Up or Sitting Down on a Chair

In order to identify a sitting or standing (from a sitting position) movement, it is instructive to note that in the natural sitting/standing movement, there are two phases in standing-to-sitting (StSi) and sitting-to-standing (SiSt) transitions:
(i) an initial leaning forwards followed by;
(ii) a leaning backwards (SiSt follows the opposite order).

In both StSi and SiSt transitions, the displacement of ⊖ (inclination angle between the trunk and vertical axis) approaches a maximum value and then recovers. A similar trend also occurs in the change in sin ⊖, as indicated in FIG. 3. Using sin ⊖ provides a non-linear scale which increases the sensitivity of the results.

That is, a StSi and SiSt postural transition (PT) is detected by analysing the pattern of sin ⊖. FIG. 3 is a graph which plots an estimation of the time at which PT of StSi or SiSt occurs (i.e. the time corresponding to the maximum of sin ⊖). The transition duration (TD) is the time interval estimated from the beginning of the leaning forward phase (P1) to the end of the leaning backward phase (P2). Hence TD=tp1−tp2, where tp1 and tp2 and are the times related to P1 and P2, which are estimated as the time corresponding to the two nearest minima, respectively. It is not necessary to determine the angle ⊖ exactly, as an estimate is sufficient for the purposes of identifying a transition. The value of θ can be estimated because the contribution of acceleration components from the posture transition can be assumed to be negligible compared to that of gravity.

Therefore, ≅ tan⁻¹(z-axis_{acceleration}/x-axis_{acceleration}).

In order to determine whether the transition is a “sit to stand” transition or a “stand to sit” transition, the Received Signal Strength Indicator (RSSI), which is the strength of the signal reflected from the WISP and detected at the antenna, is used as a method of estimating the distance of the person to the antenna and hence whether the person is standing or sitting at the end of the PT. RSSI is reported by the reader in steps of 0.5 dBm for each received signal from the WISP. A WISP at any given time will have different RSSI readings reported by different antennae and therefore each antenna is a reference point for the location and displacement of the WISP.

In more detail, after filtering to remove noise using a band pass direct-form II second-order Butterworth filter with cut-off frequencies at 0.04 and 0.7 Hz, the aforementioned three components are evaluated.

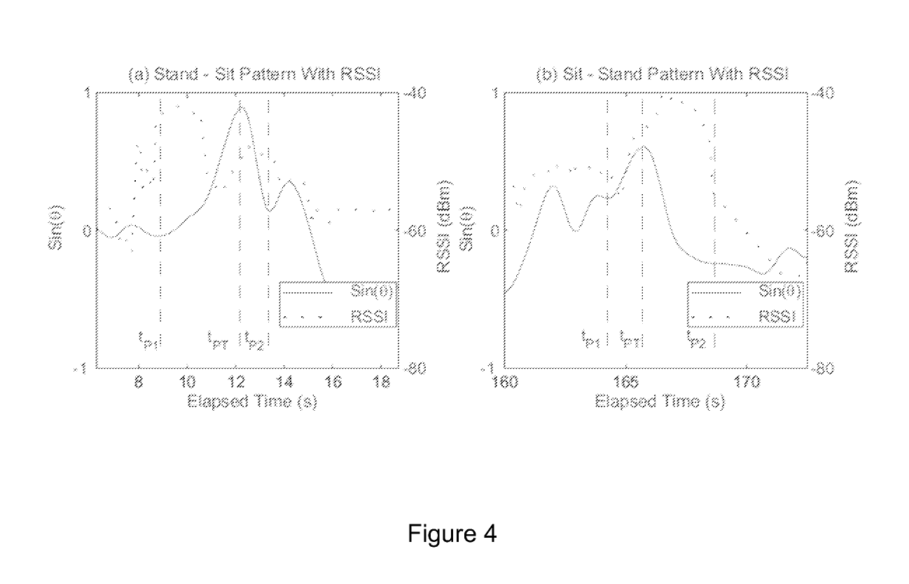

First, a true PT has a TD above 1.725 seconds and sin ⊖ larger than 0.275 at tPT. Second, the RSSI (inversely proportional to the quadruple of distance) indicates that the distance variation from the antenna due to the displacement of the body results in the RSSI reading decreasing (or increasing) depending on the location of the antenna relative to the person. As a result, when standing, the distance from the WISP to the antenna is shorter than when the person is sitting, causing a negative gradient during a StSi and positive gradient during a SiSt transition (see FIG. 4).

In short range measurements, such as within a room, RSSI can be used to successfully discriminate between SiSt and StSi transitions, as shown in FIG. 4, where the dotted line shows the RSSI values.

### Getting In and Out of Bed

A “lying down” state is determined by analysing the acceleration readings from the anteroposterior axis (xg) where readings of approximately 0 and 1 g correspond to lying and standing/sitting respectively. In practice, to eliminate noise and also reduce or remove components such as walking, the signal is filtered with a direct-form II second-order Butterworth low pass filter with cut-off frequency at 0.16 Hz.

PTs of sitting-to-lying and lying-to-sitting are detected based on threshold values before and after the event. The sitting-to-lying PT is detected using the pattern of the derivative of xg. This is the estimated time at which sitting-to-lying occurs and corresponds to the minimum of the derivative of xg while and are the times corresponding to two nearest maxima of the derivative of xg before and after tPT, respectively. The PT is classified as such if the mean of xg before and after the tPT value is above 0.7 g or below 0.4 g respectively.

### Mobilizing Without a Walking Aid

Walking is detected by analysing the vertical acceleration component every 5 seconds —the signal is filtered to distinguish the stepping patterns by isolating signals within 0.62 and 5 Hz approximately. To detect a walking period, negative peaks below a threshold of −0.05 g are considered as possible steps if 2 or more consecutive steps occur with intervals between peaks of 0.25 to 2.25 seconds.

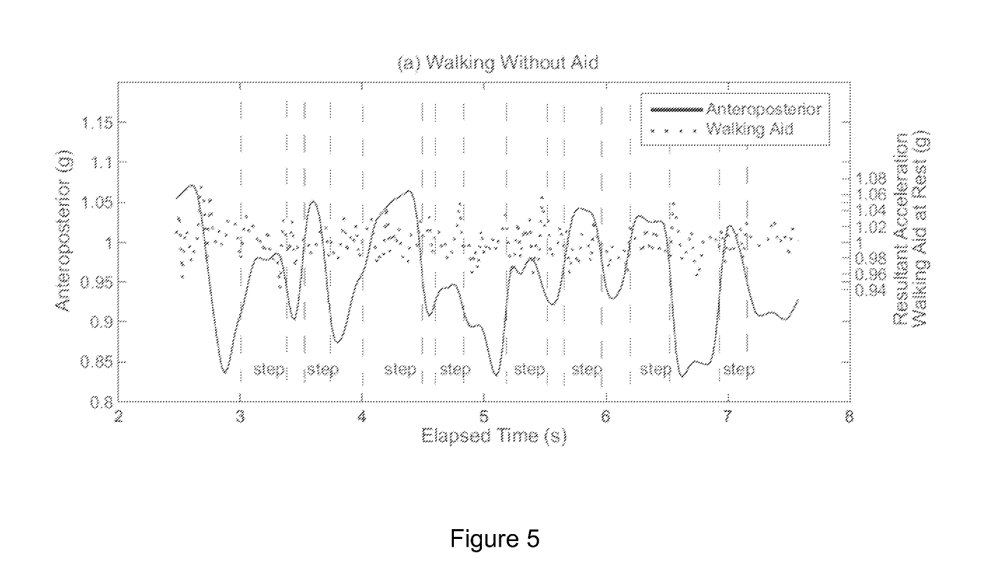

The activity of a patient walking without a walking aid is detected if a person is found to leave or enter a room or leave a position without their walking aid. A person identified as moving through a threshold without also simultaneously detecting the walking aid moving across the threshold signals the positive identification of a subject mobilizing without a walking aid. Inference is achieved by using the tag direction algorithm which indicates the direction of movement and the resultant acceleration aR reported by the WISP attached to the walking aid which in turn indicates whether the aid is being used. A value of around 1 g (gravity) confirms that the walking aid is not being used (as shown in FIG. 5) where a_{R} is given by:

a_{R}=\sqrt{aₓ²+a_{y}²+aₛ²}.

### Results

A study was conducted, using a number of volunteers, to determine the average sensitivity and specificity of the algorithms described above. The volunteers, in total, performed 197 Patient Transitions (PTs) including standing-to-sitting, sitting-to-lying, lying-to-sitting and sitting-to-standing with 99 lying conditions: supine and prone position and left and right side lying. Importantly, there were very few false positives.

Table 1, below, provides the final results from the 197 PTs performed.

TABLE 1
Results of Sensitivity and Specificity of Algorithms Utilised to Detect
Various Patient Transitions from Data Collected from WISPs

| PT | Sensitivity | Specificity |
|---|---|---|
| Sitting down on a chair (standing-to-sitting) | 92.2% | 97.9% |
| Getting up from chair (sitting-to-standing) | 90.4% | 94.0% |
| Getting into bed (sitting-to-lying) | 100% | 100% |
| Getting out of bed (lying-to-sitting) | 100% | 100% |
| Entering a room/restroom | 100% | 100% |
| Leaving a room/restroom | 100% | 100% |
| Walking without a walking aid | 100% | 100% |

As can be seen from the table, both the sensitivity and specificity of all the aforementioned algorithms and techniques is quite high, making the algorithms and techniques very suitable for the accurate determination of various movement types.

### Data Collection

Each subject was given scripted routines of postural transitions that included getting into bed, lying and getting out of bed; walking (for example walking from the bed to the chair and vice versa); and sitting down on or getting up from a chair.

Each subject was given three separate scripts with random orderings of these postural transitions. The algorithms were not customized to each subject. The transitions were recorded by the patient monitoring software and annotated simultaneously in the software system by a researcher during the data collection process. This allowed for subsequent evaluation of the results.

### Statistical Analysis

True positives were the correctly identified bed exit events (in the case of WISP on sternum algorithm, both lying to sitting followed by sitting to standing was detected correctly). True negatives were events of no interest that were correctly identified as not bed exits events (for example, getting into bed). False negatives were known bed exit events that were not identified (i.e. misses). False positives are other movements that were identified as a bed exit event. Sensitivity, and specificity, of identifying bed entry and exit was then estimated to compare the performance of the two methods. Receiver operating characteristic (ROC) curves were also evaluated.

### Results

Subjects performed over 180 PTs including standing to sitting, sitting to lying, lying to sitting and sitting to standing for the WISP attached a body trunk algorithm and 100 PTs for the algorithm based on the WISP sensor attached to mattress including, sitting, standing (implying bed empty) and lying. The results (Table 1) suggest that the WISP over the sternum method demonstrated higher sensitivity in detecting entry into and exit out of bed when compared to the WISP on mattress method. Whilst both methods recorded similar specificity in terms of detecting entry into bed, the WISP on mattress method had marginally better (97.4% vs 93.8%) specificity in terms of identification of bed exit events.

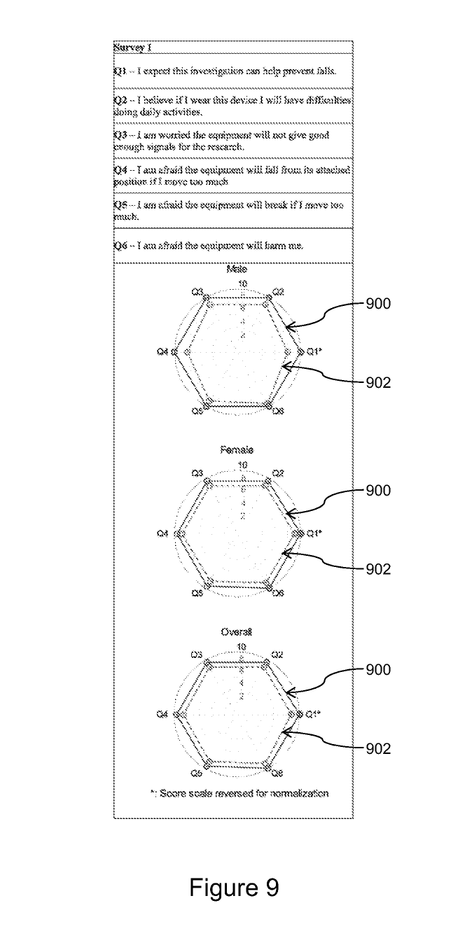

Both methods have most of their data scattered close to the left side of their graphs indicating low False Positives (i.e. false alarms) (FIG. 9). The areas under the ROC curves (AUC) were calculated by trapezoidal integration of the data. The body worn WISP AUCs were 0.931 and 0.859 for getting in and out of bed respectively and the sensor on bed algorithm had AUCs of 0.882 and 0.855 respectively. The WISP over sternum method demonstrated a better response as its curves depicted closer alignment to optimal performance (top left corner) and larger AUC for both getting in and out of bed compared to the WISP on mattress method.

Loosely fitted hospital garments may not allow the sensor to closely follow body movements affecting the effectiveness of body worn WISP algorithms to detect bed exit posture transitions. However, since the algorithms are based on thresholds and patients are automatically and uniquely identified by their electronic ID within a WISP is possible for staff to adjust the threshold levels for each patient.

### Bed-Exit Recognition Algorithm

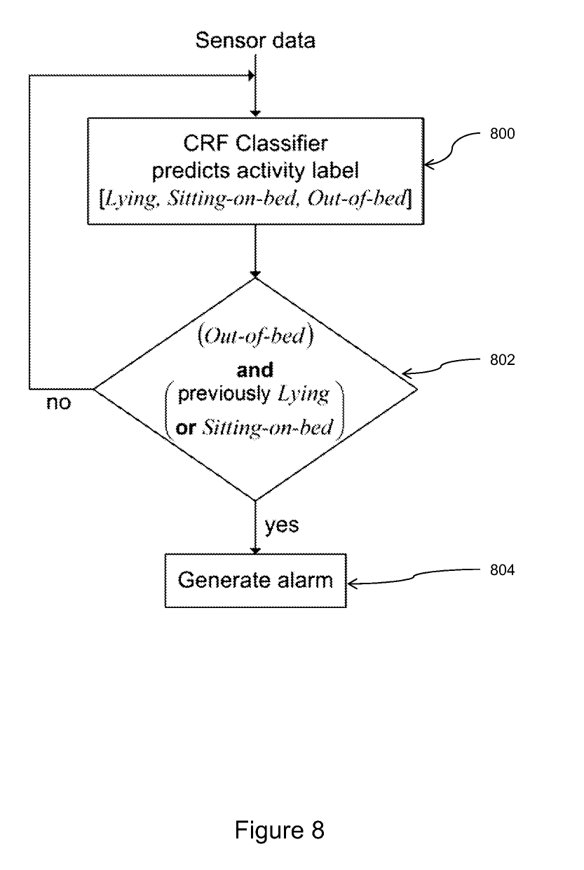

An algorithm (FIG. 8) was developed based on Conditional Random Field (CRF) learning applied in machine learning. The CRF Classifier in FIG. 8 considers an input sequence of observations to recognize multiple activities in such a sequence. Conditional models select an activity label 800 from a given set of activity labels (Lying, Sitting-on-bed, Out-of-bed) that best represents (maximizes the conditional probability) an input datum given a set of input observations. The CRF Classifier is trained using collected sensor data observations so that it is capable of predicting (since the truth about the input is unknown to the CRF Classifier) the activity label of a given input during testing of the CRF Classifier.

The raw sensor data extracted from the sensor is inputted to the algorithm without any pre-processing (such as digital filtering). Each sensor observation input to the algorithm consists of the accelerometer readings af, av and al (frontal, vertical and lateral axes respectively; with the sensor as reference), the strength of the signal received from the sensor, antenna identifier, body tilting angle with respect to the vertical given by sin(θ), where θ=arctan(a_{f}/aᵥ) and the time difference between consecutive observations. The CRF Classifier uses:
i. the strength of the signal sent from the sensor and received by RFID antennae as an indicator of relative distance or position of a participant with respect to an antenna (or bed if the antenna is located near the bed); therefore, a weaker signal is indicative of a person moving away from a given antenna (i.e. leaving the bed); and
ii. the body angle as source of information about a person's activity.

The activity model considered the following activity labels:
i. Lying;
ii. Sitting-on-bed; and
iii. Out-of-bed.

These labels correspond to the activities to be predicted (labelled) for each sensor input datum by the CRF Classifier (see FIG. 8). The bed exit recognition algorithm considers a bed exit event to be a prediction 802 of Out-of-bed label by the CRF Classifier for the current sensor datum, provided that the previous sensor datum was labelled as either Lying or Sitting-on-bed. The alert signal 804 needs to be triggered only once, i.e. only the first Out-of-bed in the sequence will trigger the signal if the previous predicted state by the CRF Classifier is either Lying or Sitting-on-bed.

### Example Study

A pilot study with 14 healthy older volunteers aged between 66 and 86 years and a male to female ratio was 2.5 was conducted. For this study, the participants were 65 years or older, living at home, able to consent to the study and mobilize independently. The subjects were recruited from geriatrician clinics and from volunteer lists from other studies. The study was completed over a two-month period where each trial with each volunteer lasted between 60 to 90 minutes.

TABLE 2
Scripted Lists Used for Performance Study Trials

| Script One | Script Two |
|---|---|
| Walk to Chair | Walk to Bed |
| Sit on Chair | Sit on Bed |
| Walk to Bed | Lay on Bed |
| Lay on Bed | Get Up and Walk to Chair |
| Sit on Bed Walk to Door | Sit on Chair |
| Walk Back to Bed | Walk to Bed |
| Lay on Bed | Lay on Bed |
| Walk to Door | Walk to Door |

### Performance Study

Participant performed activities which included:
i. lying on the bed;
ii. sitting on the bed;
iii. getting out of the bed;
iv. sitting on the chair;
v. getting out of the chair; and
vi. going from A to B (A and B represent the bed, chair or door) during the study.

Each participant performed activities on two scripted activity lists (see Table 1). No particular order was used for selecting the scripts and the number of scripted routines (where a routine is performing activities on one selected script) used in a trial was based on:
i. a participant's level of fatigue; and
ii. the trial duration where each trial period lasted no more than 90 minutes.

Participants were told to undertake the scripted activities at their own pace so as to minimise physical stress. Furthermore, the volunteers were also instructed, prior to each trial, to lie on the bed in a manner most natural and comfortable to them (i.e. no specific instruction regarding lying position was given; in the trial many participants lay down on their backs or sides, none lay in the prone position). All activities were annotated in real time by a researcher during each trial. The researcher's trialled two practical hardware deployments in two different room settings (RoomSet1 and RoomSet2 illustrated in FIG. 9) that differed in room layout (antennae placement and number of antennae deployed).

### Acceptability Study

Two surveys were designed. The first survey (administered pre and post-trial) gives an indication of a person's expectations before the trial and change in perception after the trial. The questions measured participants' perception of the system to prevent falls, their apprehension towards the use of the equipment and any changes in appreciation at the conclusion of a trial. The first survey also measured the level of motivation of the participants since a participant that is highly motivated for this investigation can influence user acceptance.

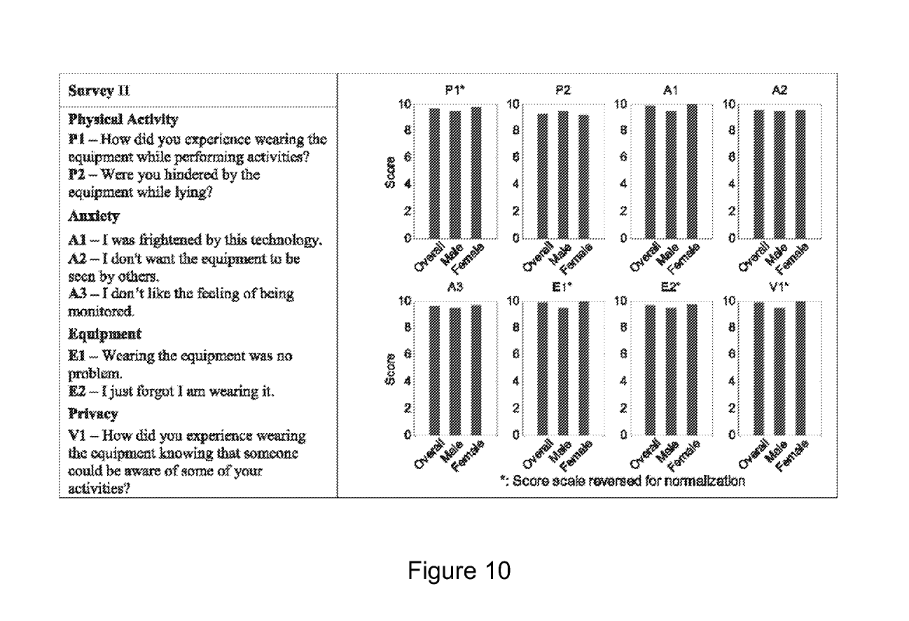

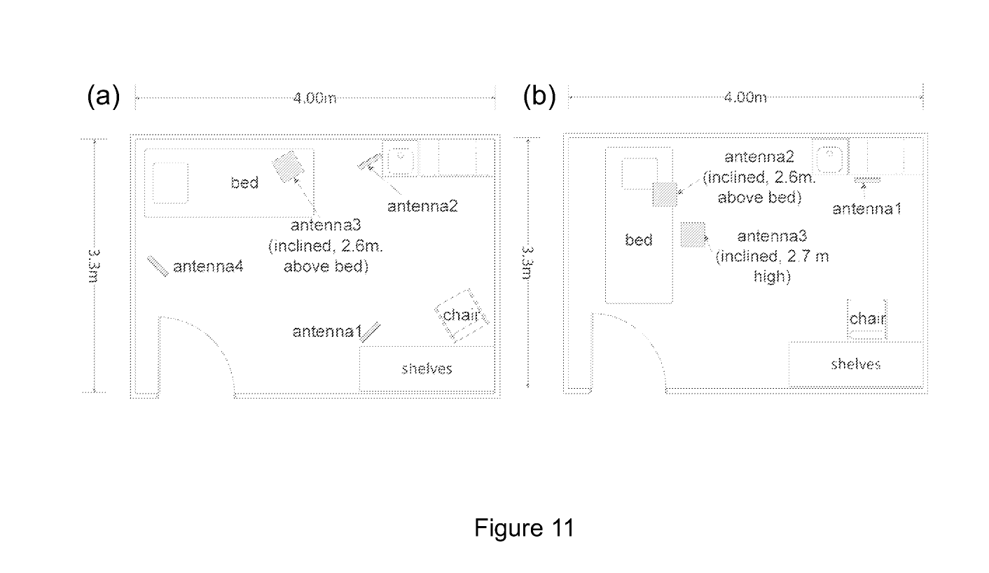

The second survey was completed after a trial concluded to measure acceptability and privacy concerns perceived by the users. The questions were formulated in positive or negative statements and used an eleven point semantic differential scale (0-10) corresponding to a completely agree to disagree or no-problem to problem range. Both surveys are shown in FIGS. 9 and 10, and responses to questions Q1, P1, E1, E2 and V1, have been reversed for a standardised meaning where a score of 10 indicates full satisfaction or conformity to the system.

### Statistical Analysis

In this study, two parameters were evaluated:
i. Sensitivity=True Positives/(True Positives+False Negatives); and
ii. Specificity=True Negatives/(True Negatives+False Positives).

True positives (TP) were correctly recognized bed exits by the classification algorithm. True negatives were activities of no-interest that were correctly identified as not bed exit events (getting into bed and lying in bed). False negatives were known bed exits that were not recognized (i.e. misses). False positives were incorrectly recognized bed exits where the person was still in bed (lying or sitting in bed).

A 10-fold cross validation which involves partitioning the sensor data set into 10 mutually exclusive subsets and validating the bed exit recognition algorithm on one subset to evaluate performance after training on the other subsets was used. The process 10 times where a particular subset was used exactly once for evaluating performance, and mean of sensitivity and specificity were determined after averaging the results of the 10 validation subsets. Data subsets used were not obtained by partitioning by a single subject or a trial but were obtained from the data sets for all participants constructed after randomly ordering data sets for scripted routines of all the participants in the study per room setting. This process ensures generalizability as well as the unbiased evaluation of the performance of the proposed bed exit recognition algorithm. The sensitivity and specificity between the two datasets (from RoomSet2, the more economical deployment, to that from RoomSet1) using an independent one-tailed t-test where statistical significance was at p-values<0.05 was compared.

TABLE 3
Sensitivity and Specificity of Bed Exit Recognition

| | RoomSet1 | | |
|---|---|---|---|
| | [mean ± SD] | RoomSet2 | RoomSet2 > RoomSet1 |
| | (%) | [mean ± SD] (%) | (p-value) |
| Sensitivity | 76.1 ± 13.6 | 90.1 ± 13.4 | 0.016 |
| Specificity | 88.1 ± 9.2 | 86.6 ± 11.1 | 0.629 |

### Results

The system collected a total of 75,108 sensor observations (readings) from both datasets and the datasets included 130 bed exits performed by 14 participants. Table 3 shows sensitivity and specificity for the two datasets. The performance in RoomSet2 was better with a higher mean sensitivity value, moreover, the sensitivity value of RoomSet2 was statistically significantly higher (p−value=0.016). However, the mean specificity values for both rooms were comparable that is RoomSet2 specificity was not statistically significantly higher (p-value=0.629). Consequently RoomSet2 is considered the better deployment configuration.

In FIG. 9 the post-trial response (solid line 900) shows a positive shift in perception (the larger outer hexagon with scores higher than the smaller inner hexagon) compared to the pre-trial response (dashed line 902). In fact, the overall score improved to ≧9.7 after the use of the system for all questions. In particular, the participants awarded maximum score post-trial to two questions (Q1 and Q6 shown in FIG. 10) corresponding to confidence in the overall system performance and its safety. In general, the male participants showed relatively lower scores than the female participants at the start of the trials but changes in perception by the male participants after the trial showed that both female and male participants felt overwhelmingly positive with similarly high scores for all questions.

Analysis of the second survey shown in FIG. 10 established the high level of acceptance of the wearable sensor based on the high scores recorded for all four factors (≧9.5 overall) of the Sensor Acceptance Model (physical activity, anxiety, equipment and privacy). The lowest score was awarded to P2, indicating a possible slight discomfort while lying, especially notable in the female participants, however the response by the female participants and the overall score was still high (>9.2).

### Software Analysis

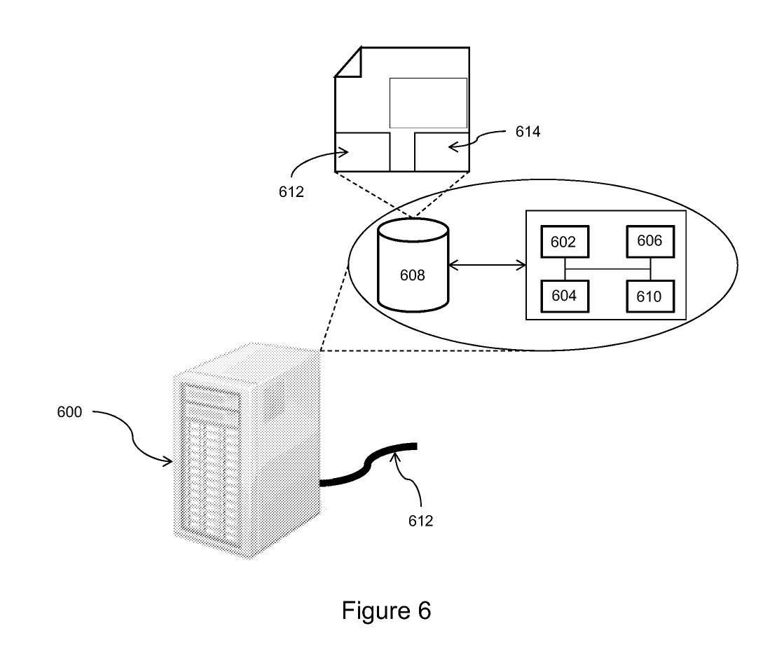

The computing system 100 of FIG. 1 is now described in more detail with reference to FIG. 6 which is a schematic diagram of a computing system 600 (equivalent to computing system 108 of FIG. 1) suitable for use as a processing module. That is, the computing system 600 may be used to execute applications and/or system services such as the monitoring software in accordance with an embodiment of the present invention.

The computing system 600 preferably comprises a processor 602, read only memory (ROM) 604, random access memory (RAM) 606, and input/output devices such as a keyboard, mouse, display and/or printer (generally denoted by 610), The computing system 600 also has one or more communications links 612. The computer includes programs that may be stored in RAM 606, ROM 604, or disk drives 608 and may be executed by the processor 602. The communications link 612 connects to a computer network such as the Internet but may be connected to a telephone line, an antenna, a gateway or any other type of communications link. Disk drives 608 may include any suitable storage media, such as, for example, floppy disk drives, hard disk drives, CD ROM drives, DVD drives or magnetic tape drives. The computing system 600 may use a single disk drive 608 or multiple disk drives. The computing system 600 may use any suitable operating systems, such as Windows™ or Unix™.

It will be understood that the computing system described in the preceding paragraphs is illustrative only, and that an embodiment of the present invention may be executed on any suitable computing system, with any suitable hardware and/or software.

In one embodiment, an example embodiment of the present invention is implemented as a software application 700 which interacts with a database 614, arranged to be executable on the computing system 600.

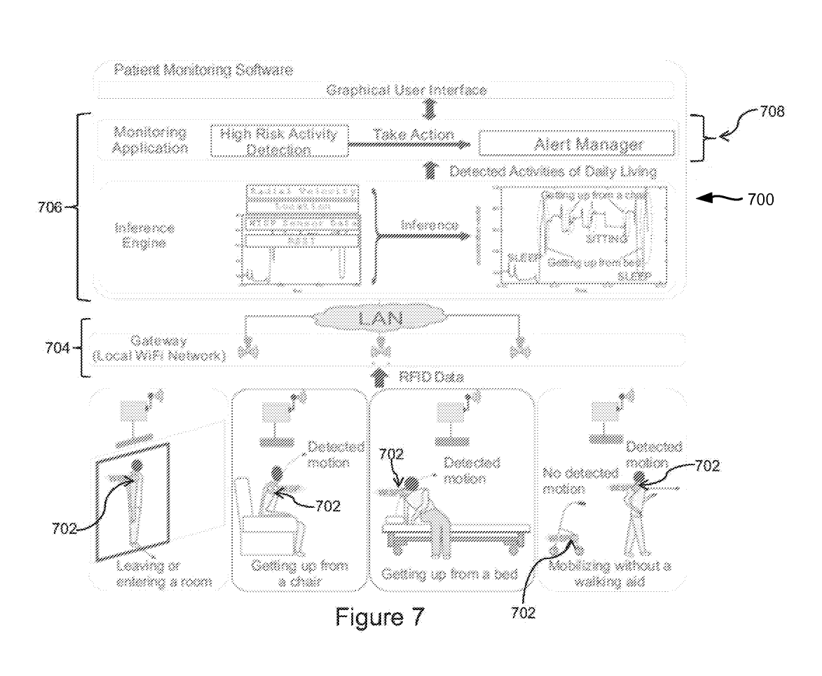

Referring to FIG. 7, the software application 700 comprises an architecture based on an event driven paradigm, where data received from the WISP devices (i.e. the passive sensors 702) via the receiving module are classified into movement types and consequently into high risk events and non-high risk events. The high risk events are then analysed by a processing module generally denoted by numeral 706. High risk events that warrant an action are then passed to the alert module.

The inference engine processes data received and collected by the RFID readers from the WISPs to identify patient activities in real-time. The interface between the Inference Engine and the RFID readers is the Low-Level Reader Protocol (EPCGlobal, Low level reader protocol (LLRP), version 1.0.1. Sensor data is gathered from the distributed network of RFID readers using the LLRP interface.

Multiple data streams (accelerometer readings, location information, direction of motion or velocity, strength of the received signal from the tags, time of event) are analysed and used to detect high risk activity by the inference engine. Then the monitoring application uses Event-Condition-Action (ECA) type rules to determine the course of action to take given high risk activity events reported by the inference engine. ECA rules are a paradigm for specifying behaviour, for example:

Rule 1: ON patient leaving room IF (no walking aid AND unsupervised) DO send alarm

In more detail, a rule based, multi-level response system is employed in the falls management system as illustrated in FIG. 6. An alerting module (the monitoring application 708) is responsible for determining whether to send an alert to caregivers based on assessing the particular high risk activity of the patient, the presence or absence of caregivers, as well as their individual assessment of falls risk recorded at the time of admittance to the hospital.

The alerting module may also include a series of rules which govern the manner in which alerts are managed. False positives and negatives will be minimized. For example, the alert module may be arranged to send an alert to a caregiver within a predefined amount of time after determining the type of risk movement.

Moreover, the alerting module may monitor the caregiver such that the alert is sent at defined intervals to the caregiver until such time as the caregiver satisfies a criterion, such as manually switching off the alert, or coming into proximity with the patient. If the primary caregiver has not responded within a preset time, the alert module may be arranged to alert a second caregiver.

The alerting module may also determine the closest caregiver and alert that caregiver even if it is not the usual caregiver to allow the fastest response time so as to prevent a fall.

The alerting module when detecting that a fall has occurred may trigger an emergency response to all caregivers in that area. It will also be understood, that in another embodiment, the alerting module may instruct an autonomous entity, such as a robot, which may then travel to location of the patient to determine whether the patient requires assistance. Alternatively, where cameras are fitted in a building, the alerting module may begin recording an image of the patient, for a caregiver to check or review to determine whether the alert is a “false positive”.

Importantly, the alerting module will be customizable to the area and meet the needs of end users. In one embodiment, the system collects identification information from the WISP to uniquely identify the person wearing the WISP. The alert module has a pre-programmed alert “profile” for each person.

For example, one person, who is more active and less likely to suffer from a serious injury, may have a reduced alert profile, such that certain movement types do not automatically trigger an alert condition. In contrast, a particularly frail person, who has a very likely to fall and suffer a serious injury, may have a high alert profile, such that any high risk movement type automatically triggers an alert condition. That is, the alert profile for each person is customisable and can be made unique to the needs of each individual.

It should be appreciated that FIGS. 6 and 7, taken singly or together, provide only one example implementation (that is, system 100 and application 700) and does not imply any limitations with regard to the environments in which different embodiments may be implemented. Many modifications to the depicted environments may be made, especially with respect to current and anticipated future advances in cloud computing, distributed computing, smaller computing devices, network communications, and the like.

In more detail, it will be understood that the algorithms used to monitor certain movements (as described above) do not, by definition, trigger an alert condition. The information derived from the algorithms can be used, in combination with other information or with knowledge about certain expected or known behaviours related to individuals and circumstances, to provide a more complex and nuanced set of conditions which need to be satisfied to trigger an alert condition. For example:

1. It is less likely that a person will suffer a fall when moving from a standing to a sitting position, than when a person is moving to a standing from a sitting position. Therefore, the alert module may be arranged to only sound an alert when a person is attempting to stand up, but not in all situations when they are attempting to sit down;

2. Similarly, a person lying down may not automatically trigger an alert condition if the person is already sitting on a bed;

3. Coming to a stationary position on the ground may be significant (i.e. such a condition may indicate a fall) and may automatically trigger an alert condition;

4. A person who is at a large distance from their walking aid may also cause an alert to be issued;

5. A person moving through a doorway to a specific room, such as a bathroom, may be considered higher risk than a person moving from their bedroom to a sitting room and therefore the location or direction of movement of a person may influence whether an alert condition is triggered;

6. A person walking or positioned for a defined period of time without an aid may also be significant and trigger an alert condition; and

7. Also, the system may be arranged to not provide an alerts during the day but to automatically provide alerts at night.

### Further Embodiment—Health Information Technology (HIT) Tool

The system described and defined above can also be integrated with a handheld Health Information Technology (HIT) tool, which can utilise data regarding patient mobility and a ‘falls’ or ‘incident’ history to automatically generate bedside posters.

### Health Information Tool

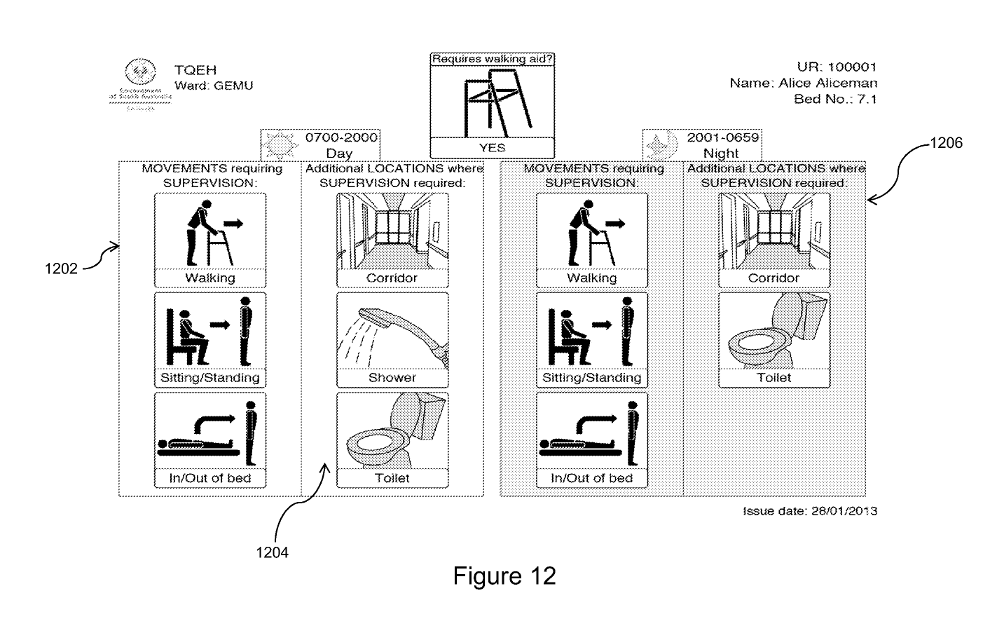

A simple and easy to use user interface design is the output desired of the bedside poster produced by the HIT tool in accordance with an embodiment of the invention. Simplified cues are provided (see FIG. 12 generally at 1202, 1204, 1206, etc.) and the number of icons is reduced by only showing those icons associated with falls risk. This simplifies the final design of the poster and reduced the amount of information that needed to be processed by viewing the poster. Moreover, the icons were selected based on the activities that might increase risk of falling.

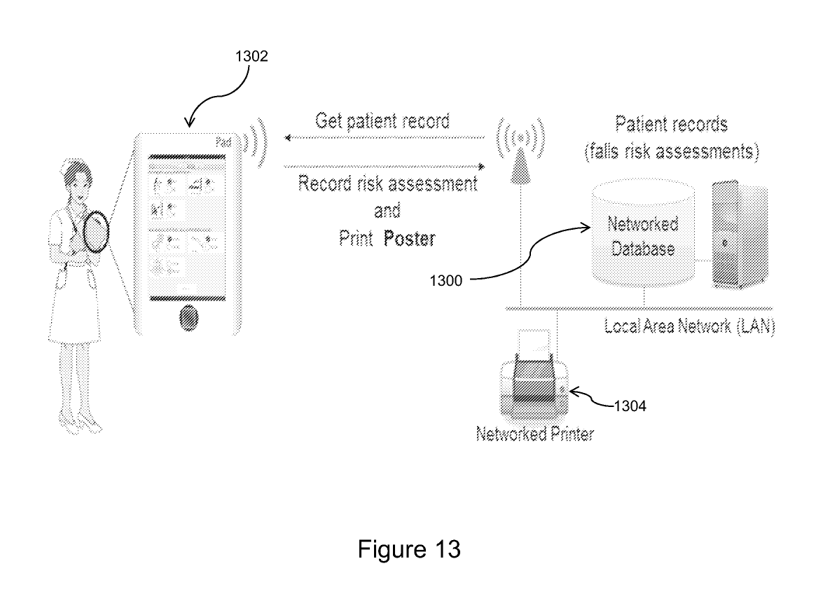

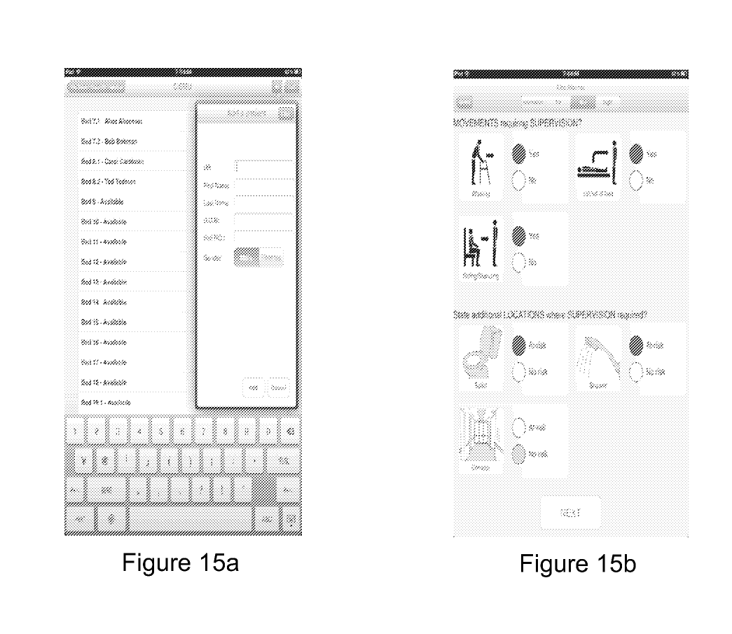

A system which interacts with the HIT tool is shown in FIG. 13. Patient falls risk assessment is stored in a database and subsequently retrieved and updated. The HIT tool 1302 automatically generates and prints the visual cues (poster) on a designated printer 1304. A detailed description of the tool designed is illustrated FIG. 13 as a workflow diagram in Unified Modelling Language (UML) whilst FIGS. 15a and 15b are screen captures of the HIT tool deployed on an iPad-mini. Following entry of risk profile into the HIT tool (see FIG. 15b), a poster for display at the bedside is printed (FIG. 12) omitting the need for the manual sticker process and enabling the integration of this clinical duty into the daily bed to bed nursing clinical handover process. Furthermore, where bedside computer are available, falls risk information can potentially be directly displayed in colour on bedside computers as opposed to the current approach of producing bedside posters.

### HIT Tool Internal Logic

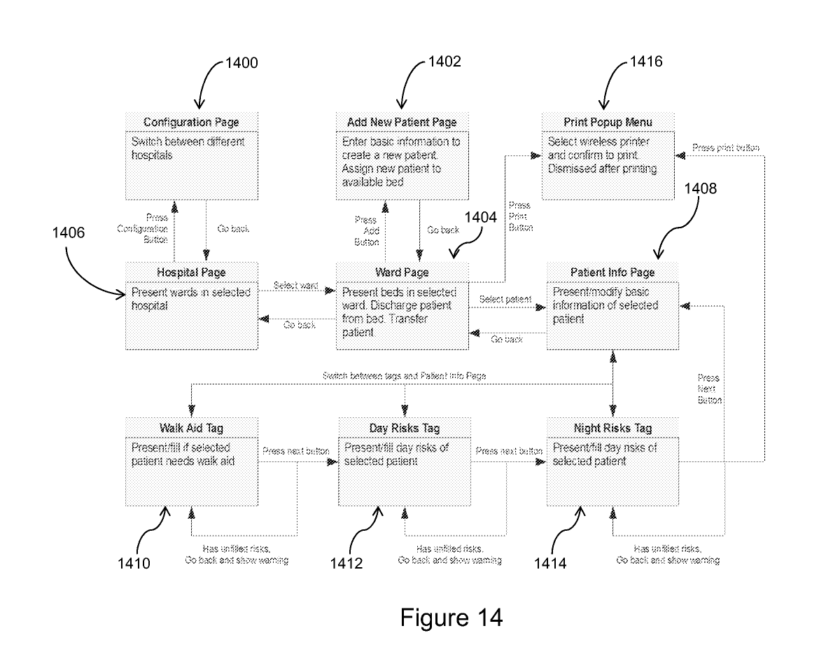

Referring to FIG. 14, there is shown a process flow in accordance with an embodiment of the HIT tool described herein. At 1400 there is shown a configuration page which allows a user to switch between different hospitals. At 1402 there is shown a new patient page which allows a user to enter basic information to create a new patient or to assign a new patient to an available bed. At 1404 there is shown a ward page which allows a user to present beds in selected wards, discharge patients from a bed or transfer patients. At 1406 there is shown a hospital page which allows a user to present wards in a selected hospital. At 1408 there is shown a patient info page which allows a user to present/modify basic information of the selected patient. At 1410, 1412 and 1414 there are shown respective tags which allow patients to be tagged with certain information. For example, at 1410 a patient can be tagged with a walk aid tag if the patient requires a walk aid. If the patient presents a risk during the day, the patient may be tagged as a day risk 1412, and similarly if the patient presents a risk at night, the patient may be tagged as a night risk at 1414. As can be seen by the arrows in FIG. 14, a user may navigate through the various options and may “add” or “remove” patients, assign patients to particular wards or hospitals, and tag patients as having certain requirements. These requirements can then be translated and automatically printed into the label shown generally at FIG. 12. Printing is achieved by the print “pop-up” menu which is shown generally at 1416 in FIG. 14.

### The Survey

In order to evaluate the usability and nursing staff perception of the handheld Health Information Technology (HIT) tool the Questionnaire for User Interface Satisfaction (QUIS) was modified for use in this study by adding six questions (Q19 to Q24) (Table I) to evaluate staff perception of the technology in addition to the usability of the HIT tool. Staff provided responses to likert scales with scores between 0 and 9 and in this study the researchers opted to consider 0-3 as a negative response and 7-9 as a positive response. Scores in between were classified as ambivalence. Scores are also presented as mean and standard error of mean (SEM).

### Study Participants

The survey occurred at the Queen Elizabeth Hospital in Australia, a 300 bed general hospital. Nursing staff members from two medical wards (Geriatric Evaluation and Management Unit and Acute Medical Unit) were approached to participate between the hours of 0900 and 1700 over two days in January 2013.

### Results

Responses to the modified QUIS survey were obtained from 25 nurses with a mean age of 40.9 years (standard deviation 11.8) and an average of 14.9 years (SD 10.9) working experience. Survey results are presented as percentages or mean and standard deviation (SD). The proportions of positive responses (score of 7-9) to survey questions are reported in Table 4.

TABLE 4
Summary of Nursing Survey Results

| | | |
|---|---|---|
| | Posi- | |
| Modified Questionnaire | tive | Mean |
| for User Interface Satisfaction | (%) | (SEM)* |

Appearance of Screen

| | | | |
|---|---|---|---|
| Q1 | Wonderful | 80.0 | 7.24 (0.28) |
| Q2 | Easy | 88.0 | 7.64 (0.28) |
| Q3 | Satisfying | 76.0 | 7.20 (0.29) |
| Q4 | Stimulating | 60.0 | 7.04 (0.23) |
| Q5 | Flexible | 76.0 | 7.24 (0.23) |
| Q6 | Characters on screen easy to read | 96 | 8.24 (0.18) |

Ease of Use

| | | | |
|---|---|---|---|
| Q7 | Highlighting on screen simplifies task very | 88.0 | 7.80 (0.36) |
| | much | | |
| Q8 | Organization of information on screen very | 92.0 | 7.88 (0.19) |
| | clear | | |
| Q9 | Sequence of screens very clear | 92 | 7.88 (0.19) |
| Q10 | Use of terms throughout the system | 92 | 7.76 (0.21) |
| | consistent | | |
| Q11 | Terms are always related to the task I am | 87 | 7.52 (0.25) |
| | doing | | |
| Q12 | Messages on screen which prompt user for | 95.8 | 7.79 (0.24) |
| | input clear | | |
| Q13 | Error message helpful | 82.6 | 7.43 (0.27) |
| Q14 | Learning to operate the system easy | 91.7 | 7.96 (0.26) |
| Q15 | Tasks can always be performed in a | 91.7 | 7.92 (0.19) |
| | straightforward manner | | |
| Q16 | System speed fast enough | 91.7 | 8.00 (0.19) |
| Q17 | Correcting mistakes easy | 82.6 | 7.30 (0.41) |
| Q18 | Experienced and inexperienced user's needs | 91.7 | 7.63 (0.24) |
| | are always taken into consideration | | |

Additional Questions Addressing Staff Perception
of Usability And Benefits

| | | | |
|---|---|---|---|
| Q19 | I feel that this tool will be easy to use during | 94.7 | 7.84 (0.28) |
| | the bed to bed handover or on ward rounds | | |
| Q20 | I feel that this tool will help me provide safer | 78.9 | 7.53 (0.33) |
| | care for patients at risk of falls | | |
| Q21 | I feel this tool will assist me update | 94.7 | 7.84 (0.26) |
| | information about the patient's risk in a | | |
| | timely manner | | |
| Q22 | I think the tool will be quicker in terms of | 89.5 | 7.95 (0.27) |
| | updating the information and preparing | | |
| | poster/visual cue for display at bedside | | |
| | compared to the current method | | |
| Q23 | I feel the tool will improve quality of patient | 84.2 | 7.48 (0.32) |
| | care | | |
| Q24 | I will use this tool if it is made available | 94.7 | 8.21 0.25) |

*SEM—Standard Error of the Mean.

For all questions, the mean score was 7 or more. A large proportion of nursing staff responded positively to other aspects of the tool (78% to 94.7%). One of the key finding was that the surveyed population of nurses overwhelmingly agreed (highest positive response of 94.7% for Q24) that the HIT tool was useful. Nursing staff also confirmed that they felt that the tool could be incorporated into their clinical handover process to aid them with their work and to improve the safety and quality of care provided to patients (Q19 and Q21, >94%).

The study confirms that nursing staff felt positive about the HIT tool and indicated a strong likelihood of using the tool and integrating it into their daily clinical bed to bed handover process. Although falls risk assessment using standardized falls risk assessment tools exist, clinicians have previously reported difficulties in effectively communicating the falls risk status to each other as well as to patients and their family and this is where visual cues can potentially play a role. The HIT tool improves the completion rate as well as accuracy of bedside posters within the hospital and integrates seamlessly with the system method, and software application and data signal for determining movement broadly described herein.

### Advantages

The embodiment described herein provides a number of advantages over known devices and techniques. The embodiment simplifies real-time monitoring of persons who are engaging in any type of movement, but finds particular application in the monitoring of persons who are at risk of injuring themselves.

In other words, the system, methods and software application embodiments described herein, and the broader inventive concepts defined in the claims, provide a technological intervention to monitor patient mobility and therefore prevent falls in many high risk environments, such as hospitals and aged care facilities.

Advantageously, the system described herein utilises passive, battery-less WISP devices which are cheap to manufacture, add no burden or weight to the patient (due to their small size and insignificant weight) and have very high sensitivity and specificity rates, as previously described.

The system, method and software application largely ameliorate traditional sensor based systems, which require battery power and are therefore heavier, more expensive, more prone to failure and less accurate than the embodiments described herein.

Additionally, the system is customizable to individual patients and care environments and automatically determines the level of monitoring and care required for each patient based on the expert knowledge of clinicians or caregivers.

Moreover, the system is capable of being used to detect the presence and/or movement of inanimate objects, such as canes, walking sticks, walking frames, etc. By utilising a combination of devices on both persons and objects, sophisticated and complex actions (such as whether a person is using a walking aid) can be easily and accurately detected.

The present invention, in its various embodiments, configurations, and aspects, includes components, methods, processes, systems and/or apparatuses substantially as depicted and described herein, including various embodiments, sub-combinations, and subsets thereof. Those of skill in the art will understand how to make and use the present invention after understanding the present disclosure. The present invention, in its various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease and\or reducing cost of implementation.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments, configurations, or aspects for the purpose of streamlining the disclosure. The features of the embodiments, configurations, or aspects of the invention may be combined in alternate embodiments, configurations, or aspects other than those discussed above. This method of disclosure is not to be interpreted as reflecting an intention that the claimed invention requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the invention.

Moreover, though the description of the invention has included description of one or more embodiments, configurations, or aspects and certain variations and modifications, other variations, combinations, and modifications are within the scope of the invention, e.g., as may be within the skill and knowledge of those in the art, after understanding the present disclosure. It is intended to obtain rights which include alternative embodiments, configurations, or aspects to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

The flowchart and block diagrams in the above-described figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The embodiments described herein may be implemented in digital electronic circuitry, or in computer hardware, firmware, software, or in combinations of them. The embodiments can be implemented as a computer program product, i.e., a computer program tangibly embodied in an information carrier, e.g., in a machine-readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computer system. A computer program can be deployed to be executed on one computer or on multiple computers at one site or distributed across multiple sites and interconnected by a communication network.

Method or function steps of the embodiments described herein can be performed by one or more programmable processors executing a computer program to perform functions of the invention by operating on input data and generating output. Method or function steps can also be performed by, and system and/or apparatus of the invention can be implemented as, special purpose logic circuitry, e.g., an FPGA (field programmable gate array) or an ASIC (application-specific integrated circuit). Modules may refer to portions of the computer program and/or the processor/special circuitry that implements that functionality.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

To provide for interaction with a user, the embodiments described herein can be implemented on a computer having a display device, e.g., a CRT (cathode ray tube) LED (light emitting diode), or LCD (liquid crystal display) monitor, for displaying information to the user and a keyboard and a pointing device, e.g., a mouse or a trackball, by which the user can provide input to the computer (e.g., interact with a user interface element, for example, by clicking a button on such a pointing device). Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The embodiments described herein may further be implemented in a distributed computing system that includes a back-end component, e.g., as a data server, and/or a middleware component, e.g., an application server, and/or a front-end component, e.g., a client computer having a graphical user interface and/or a Web browser through which a user can interact with an implementation of the invention, or any combination of such back-end, middleware, or front-end components. The components of the system can be interconnected by any form or medium of digital data communication, e.g., a communication network. Examples of communication networks include a LAN and WAN, e.g., the Internet, and include both wired and wireless networks.

The computing system can include clients and servers. A client and server are generally remote from each other and typically interact over a communication network. The relationship of client and server arises by virtue of computer programs running on the respective computers and having a client-server relationship to each other.

Although the present invention describes components and functions implemented in the embodiments with reference to particular standards and protocols, the invention is not limited to such standards and protocols. Other similar standards and protocols not mentioned herein are in existence and are considered to be included in the present invention. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present invention.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

It is to be understood that the foregoing description is intended to illustrate and not to limit the scope of the invention, which is defined by the scope of the appended claims. Other embodiments are within the scope of the following claims.

1.-65. (canceled)

66. A computer system for determining movement, comprising:
a processing hardware set, and
a computer readable storage device medium, wherein
the processing hardware set is structured, connected and/or programmed to run program instructions stored on the computer readable storage medium, the program instructions including:
at least one receiving module adapted to receive movement data indicative of movement from a remote device,
at least one processing module adapted to process the moving data to determine a type of movement, and
at least one alerting module adapted to provide an alert in the event that the type of movement falls within at least one predetermined type.

67. The system of claim 66, wherein
the movement data includes acceleration data, and
the movement data is utilized to calculate a motion vector indicative of the movement of the remote device.

68. The system of claim 67, wherein the type of movement is determined, in part, by analyzing variations in the motion vector over a period of time.

69. The system of claim 66, wherein the at least one receiving module is adapted to receive movement data from a plurality of remote devices and incorporates a radiofrequency signal emitter arranged to send an activation signal to the remote device.

70. The system of claim 66, wherein the at least one receiving module is adapted to receive the movement data as a radiofrequency signal, and wherein the remote device is a passive radiofrequency device adapted to emit a radiofrequency signal encoding the movement data upon exposure to the activation signal.

71. The system of claim 66, wherein the predetermined types of movements include:
movements by a person or object, and
movements likely to cause injury and movements likely to carry a high risk of injury when performed.

72. The system of claim 71, wherein the at least one processing module is further adapted to:
determine whether the type of movement corresponds to one of the predetermined movement types,
calculate angular displacement of the remote device relative to a predefined axis over a defined period of time, and
determine whether the angular displacement of the remote device increases to a maximum value from a base level and subsequently returns to the base level.

73. The system of claim 71, wherein the at least one processing module selects a movement type from a group including walking through a doorway, sitting, standing, lying, getting up from lying and walking without a walking aid.

74. The system of claim 66, wherein the at least one processing module is further adapted to:
calculate a radial velocity of the at least one remote device over a defined period of time,
determine whether the direction of the radial velocity has changed over the defined period of time, and
classify the type of movement as walking through a doorway when the direction of the radial velocity has changed over the defined period of time.

75. The system of claim 72, wherein the at least one processing module is further adapted to calculate an acceleration component in one predefined axis over a defined period of time, the predefined axis being one of an axis substantially vertical relative to a ground surface or an axis substantially horizontal relative to a ground surface.

76. The system of claim 75, wherein the at least one processing module is further adapted to:
analyze the acceleration component over a period of time to determine whether a pattern exists, and
classify the type of movement as walking without a walking aid when the pattern of a first device does not correspond with the pattern of a second device.

77. The system of claim 72, wherein the at least one processing module is further adapted to determine whether the angular displacement of the at least one remote device increases to a maximum value from a base level and subsequently returns to the base level.

78. The system of claim 77, wherein the at least one processing module is further adapted to classify the type of movement as sitting when the angular displacement has changed over the defined period of time to the maximum state and subsequently returns to a base level.

79. The system of claim 66, wherein
the at least one alerting module sends an alert to a person at defined intervals to the person until such time as the person satisfies a criterion, and
the system maintains a record of the amount of time elapsed between sending of the alert to the person and the time at which the person satisfies the criterion.

80. The system of claim 79, wherein the criterion includes locating of a first remote device in proximity with a second remote device.

81. The system of claim 79, wherein
the at least one receiving module receiving identification data from the remote device,
the at least one alerting module utilizes the identification data to, in part, determine the alert condition.

82. A method for determining movement, comprising:
receiving movement data indicative of movement from at least one remote device,
processing the movement data to determine the type of movement, and
providing an alert in the event that the type of movement falls within at least one predetermined type, the movement data including acceleration data and useable to calculate a motion vector indicative of the movement of the remote device,
wherein the receiving, processing, and providing steps are performed by computer software adapted to run on computer hardware.

83. The method of claim 82, wherein the type of movement is determined, in part, by analyzing variations in the motion vector over a period of time.

84. The method of claim 82, wherein receiving further includes receiving movement data from a plurality of remote devices.

85. The method of claim 82, wherein the predetermined types of movements include:
movements by a person or object, and
movements likely to cause injury and movements likely to carry a high risk of injury when performed.

86. A non-transitory computer readable information storage media having stored thereon information, that when executed by a processor, causes to be performed the steps in claim 82.

87. A set of machine readable instructions and associated data, stored on a storage device in a manner more persistent than a signal in transit, the set comprising:
at least one receiving module programmed to receive movement data indicative of movement from a remote device,
at least one processing module programmed to process the moving data to determine a type of movement, and
at least one alerting module programmed to provide an alert in the event that the type of movement falls within at least one predetermined type.
